(19) United States
(12) Patent Application Publication
(10) Pub. No.: US 2025/0017616 A1
(43) Pub. Date: Jan. 16, 2025

(54) THROMBUS REMOVAL SYSTEMS AND ASSOCIATED METHODS

(71) Applicant: Shifamed Holdings, LLC, Campbell, CA (US)

(72) Inventor: Uday ILLINDALA, Los Gatos, CA (US)

(21) Appl. No.: 18/773,208

(22) Filed: Jul. 15, 2024

Related U.S. Application Data
(60) Provisional application No. 63/513,531, filed on Jul. 13, 2023.

(51) Int. Cl. A61B 17/3203; A61B 17/00

(52) U.S. Cl. CPC A61B 17/32037; A61B 2017/00778

(57) ABSTRACT

The present technology relates to systems and methods for removing a thrombus from a blood vessel of a patient. In some embodiments, the present technology is directed to systems including an elongated catheter having a distal portion configured to be positioned within the blood vessel of the patient, a proximal portion configured to be external to the patient, and a lumen extending therebetween. The system can also include a fluid delivery mechanism coupled with a fluid lumen and configured to apply fluid to at least partially fragment the thrombus.

### CLAIM OF PRIORITY

This application claims the benefit of U.S. Provisional Application No. 63/513,531, filed Jul. 13, 2024.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present technology generally relates to medical devices and, in particular, to systems including aspiration and fluid delivery mechanisms and associated methods for removing a thrombus from a mammalian blood vessel.

### BACKGROUND

Thrombotic material may lead to a blockage in fluid flow within the vasculature of a mammal. Such blockages may occur in varied regions within the body, such as within the pulmonary system, peripheral vasculature, deep vasculature, or brain. Pulmonary embolisms typically arise when a thrombus originating from another part of the body (e.g., a vein in the pelvis or leg) becomes dislodged and travels to the lungs. Anticoagulation therapy is the current standard of care for treating pulmonary embolisms, but may not be effective in some patients.

Additionally, conventional devices for removing thrombotic material may not be capable of navigating the tortuous vascular anatomy, may not be effective in removing thrombotic material, and/or may lack the ability to provide sensor data or other feedback to the clinician during the thrombectomy procedure. Existing thrombectomy devices operate based on simple aspiration which works sufficiently for certain clots but is largely ineffective for difficult, organized clots. Many patients presenting with deep clots in difficult to reach anatomical locations and/or deep vein thrombus (DVT) or PE are left untreated as long as the risk of limb ischemia is low.

In more urgent cases, they are treated with catheter-directed thrombolysis or lytic therapy to break up a clot over the course of many hours or days.

More recently other tools like clot retrievers have been developed to treat DVT and pulmonary embolism (PE). Clot retrievers typically include a structure that is deployed from a distal end of the catheter within the vessel to capture thrombus and then withdrawn back into the distal end of the catheter for thrombus removal. The structure can include stent-like structures, expandable capture baskets, or capture structures that include passive capture features like rakes, barbs, or prongs to engage the clot. These tools are not being widely adopted because of their limited effectiveness, high mortality rates, and additional costs versus aspiration or the standard of care. Additionally, advancing the capture structure distally from the end of the catheter poses additional challenges including limited visualization of the clot relative to the capture device and the risk of damaging vessel walls with the passive capture structures. Other recent developments focus on slicing or macerating the clot, but these mechanisms are designed to reduce the risk of the catheter clogging and do not address the problem of tough, large, organized clots. There remains the need for a device to address these and other problems with existing venous thrombectomy including, but not limited to, a fast, easy-to-use, and effective device for removing a variety of clot morphologies in difficult to reach anatomical locations.

Right ventricular (RV) function is a major determinant of morbidity and mortality for a variety of cardiovascular diseases, but RV function is challenging to characterize and quantify. 2D imaging modalities struggle to negotiate the ventricle's irregular position in the chest and its asymmetrical geometry, and as a result can only characterize contractile function in a single direction or from a particular aspect of the ventricle. More sophisticated modalities like cardiac magnetic resonance (CMR) and 3D echocardiographic imaging, while able to overcome some of these limitations, provide predominantly volume-centric descriptions of RV function.

Pressure-volume (PV) analysis addresses these shortcomings by combining simultaneous measurements of pressure and volume to generate load-independent measures of systolic and diastolic chamber properties to characterize the ventricular systolic and diastolic function, as well as ventricular-vascular interactions.

### SUMMARY OF THE DISCLOSURE

A method, comprising: advancing a thrombectomy system into a pulmonary artery of a subject; periodically or continuously measuring a pulmonary artery pressure with the thrombectomy system; advancing a P-V catheter into a right ventricle of the subject; periodically or continuously measuring right ventricular pressure, right ventricular conductance, and/or a right ventricular admittance with the P-V catheter; initiating a thrombectomy procedure with the thrombectomy system; and determining a treatment progress or treatment completion state based on a correlation between the measured pulmonary artery pressure and/or the right ventricular pressure, right ventricular conductance, and/or a right ventricular admittance.

In some aspects, the method includes providing an output related to the treatment progress or treatment completion state to a user of the thrombectomy system.

In one aspect, the method includes performing a pressure-volume loop analysis with the right ventricular pressure, right ventricular conductance, and/or a right ventricular admittance.

In some aspects, the correlation is based on a relationship between vascular function and right ventricular function.

In one aspect, the method includes determining the treatment progress or treatment completion state algorithmically based on a change in a measured parameter exceeding a predetermined threshold.

In another aspect, the method includes determining the treatment progress or treatment completion state with a machine learning model.

In some aspects, the machine learning model is trained by tagging the treatment progress or treatment completion state with one or more training data sets.

In one aspect, the output comprises a label of treatment progress.

In some aspects, the label is selected from the group consisting of not complete, partially complete, and treatment complete.

In another aspect, the label is an indicator of treatment progress.

In some aspects, the label is a percentage of treatment completion.

A method is provided, comprising: advancing a thrombectomy system into a pulmonary artery of a subject; periodically or continuously measuring a pulmonary artery pressure with the thrombectomy system; advancing a P-V catheter into a right ventricle of the subject; periodically or continuously measuring right ventricular pressure, right ventricular conductance, and/or a right ventricular admittance with the P-V catheter; initiating a thrombectomy procedure with the thrombectomy system; evaluating onboard catheter data; and determining a system state based on a correlation between the onboard data and/or measured pulmonary artery pressure and/or the right ventricular pressure, right ventricular conductance, and/or a right ventricular admittance.

In some aspects, the method comprises providing an output of the system state to a user of the thrombectomy system.

In one aspect, the method further comprises performing a pressure-volume loop analysis with the right ventricular pressure, right ventricular conductance, and/or a right ventricular admittance.

In some aspects, the correlation is based on a relationship between vascular function and right ventricular function.

In other aspects, the method includes determining the system state algorithmically based on a change in onboard catheter data exceeding a predetermined threshold.

In some aspects, the method includes determining the system state with a machine learning model.

In some aspects, the machine learning model is trained by tagging the system state with one or more training data sets.

In other aspects, the system state comprises a label that describes clot engagement.

In one aspect, the label is selected from the group consisting of clear, partially engaged, and engaged.

A thrombectomy system is provided, comprising: an introducer sheath; a thrombectomy device adapted to be inserted into the introducer sheath to place the thrombectomy device within a pulmonary artery of a subject, the thrombectomy device including an aspiration lumen coupled to an aspiration source; a pressure sensor disposed on the introducer sheath and/or the thrombectomy device and configured to measure a pulmonary artery pressure of the subject; a P-V catheter adapted to be inserted into a right ventricle of the subject, the P-V catheter being configured to measure a right ventricular pressure, a right ventricular conductance, and/or a right ventricular admittance; one or more processors; and memory coupled to the one or more processors, the memory configured to store computer-program instructions, that, when executed by the one or more processors, implement a computer-implemented method, the computer-implemented method comprising: determining a treatment progress or treatment completion state based on a correlation between the measured pulmonary artery pressure and/or the right ventricular pressure, right ventricular conductance, and/or a right ventricular admittance.

A thrombectomy system is provided, comprising: an introducer sheath; a thrombectomy device adapted to be inserted into the introducer sheath to place the thrombectomy device within a pulmonary artery of a subject, the thrombectomy device including an aspiration lumen coupled to an aspiration source; a pressure sensor disposed on the introducer sheath and/or the thrombectomy device and configured to measure a pulmonary artery pressure of the subject; a P-V catheter adapted to be inserted into a right ventricle of the subject, the P-V catheter being configured to measure a right ventricular pressure, a right ventricular conductance, and/or a right ventricular admittance; one or more processors; and memory coupled to the one or more processors, the memory configured to store computer-program instructions, that, when executed by the one or more processors, implement a computer-implemented method, the computer-implemented method comprising: determining a system state based on a correlation between onboard data and/or the measured pulmonary artery pressure and/or the right ventricular pressure, right ventricular conductance, and/or a right ventricular admittance.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1-1L illustrate various views of a portion of a thrombus removal system including a distal portion of an elongated catheter configured in accordance with an embodiment of the present technology.

FIGS. 2A-2E illustrate plan views of various configurations of irrigation ports and fluid streams of a thrombus removal system according to embodiments of the present technology.

FIGS. 3A-3H illustrate an elevation view of various configurations of irrigation ports and fluid streams of a thrombus removal system according to embodiments of the present technology.

FIGS. 4A-4C illustrate various embodiments of a thrombus removal system including a saline source, an aspiration system, and one or more controls for controlling irrigation and/or aspiration of the system.

FIGS. 5A-5K illustrate a thrombectomy method that can include performing PV loop analysis.

FIG. 6 is an example of a pressure-volume loop analysis.

### DETAILED DESCRIPTION

This application is related to disclosure in International Application No. PCT/US2021/020915, filed Mar. 4, 2021 (the '915 application), and International Application No. PCT/US2022/033024, filed Jun. 10, 2022 (the '024 application), the disclosures of which are incorporated by reference herein for all purposes. The '915 and '024 applications describe general mechanisms for capturing and removing a clot. By example, multiple fluid streams are directed toward the clot to fragment the material.

The present technology is generally directed to thrombus removal systems and associated methods. A system configured in accordance with an embodiment of the present technology can include, for example, an elongated catheter having a distal portion configured to be positioned within a blood vessel of the patient, a proximal portion configured to be external to the patient, a fluid delivery mechanism configured to fragment the thrombus with pressurized fluid, an aspiration mechanism configured to aspirate the fragments of the thrombus, and one or more lumens extending at least partially from the proximal portion to the distal portion.

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples but are not described in detail with respect to the figures.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “generally,” “approximately,” and “about” are used herein to mean the stated value plus or minus 10%.

Although some embodiments herein are described in terms of thrombus removal, it will be appreciated that the present technology can be used and/or modified to remove other types of emboli that may occlude a blood vessel, such as fat, tissue, or a foreign substance. Additionally, although some embodiments herein are described in the context of thrombus removal from a pulmonary artery (e.g., pulmonary embolectomy), the technology may be applied to removal of thrombi and/or emboli from other portions of the vasculature (e.g., in neurovascular, coronary, within chambers of the heart, or peripheral applications). Moreover, although some embodiments are discussed in terms of maceration of a thrombus with a fluid, the present technology can be adapted for use with other techniques for breaking up a thrombus into smaller fragments or particles (e.g., ultrasonic, mechanical, enzymatic, etc.).

The headings provided herein are for convenience only and do not interpret the scope or meaning of the claimed present technology.

### Systems for Thrombus Removal

As provided above, the present technology is generally directed to thrombus removal systems. Such systems include an elongated catheter having a distal portion positionable within a blood vessel of the patient (e.g., an artery or vein), a proximal portion positionable outside the patient's body, a fluid delivery mechanism configured to fragment the thrombus with pressurized fluid, an aspiration mechanism configured to aspirate the fragments of the thrombus, and one or more lumens extending at least partially from the proximal portion to the distal portion. In some embodiments, the systems herein are configured to engage a thrombus in a patient's blood vessel, break the thrombus into small fragments, and aspirate the fragments out of the patient's body. The pressurized fluid streams (e.g., jets) function to cut or macerate thrombus, before, during, and/or after at least a portion of the thrombus has entered the aspiration lumen or a funnel of the system. Fragmentation helps to prevent clogging of the aspiration lumen and allows the thrombus removal system to macerate large, firm clots that otherwise could not be aspirated. As used herein, “thrombus” and “embolism” are used somewhat interchangeably in various respects. Typically a thrombus is a portion of clotted blood that has stopped moving through the vasculature and is lodged or stuck and the emboli is a portion of clotted blood that is moving in the vasculature that can eventually become a thrombus and additionally seed a larger thrombus either by collecting other emboli or blood clotting on the thrombus.

It should be appreciated that while the description may refer to removal of “thrombus,” this should be understood to encompass removal of thrombus fragments and other emboli as provided herein.

According to embodiments of the present technology, a fluid delivery mechanism can provide a plurality of fluid streams (e.g., jets) to fluid apertures of the thrombus removal system for macerating, cutting, fragmenting, pulverizing and/or urging thrombus to be removed from a proximal portion of the thrombus removal system. The thrombus removal system can include an aspiration lumen extending at least partially from the proximal portion to the distal portion of the thrombus removal system that is adapted for fluid communication with an aspiration pump (e.g., vacuum source). In operation, the aspiration pump may generate a volume of lower pressure within the aspiration lumen near the proximal portion of the thrombus removal system, urging aspiration of thrombus from the distal portion to the proximal portion.

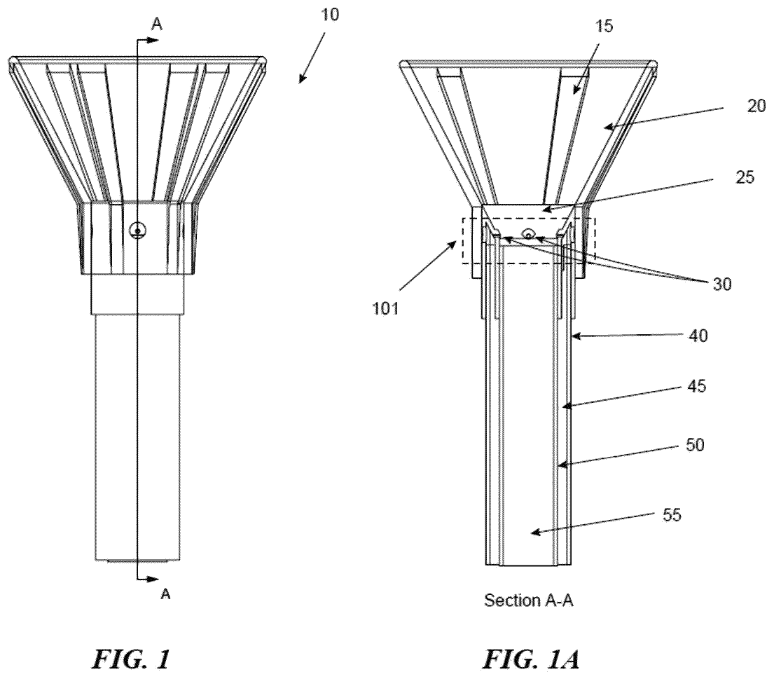

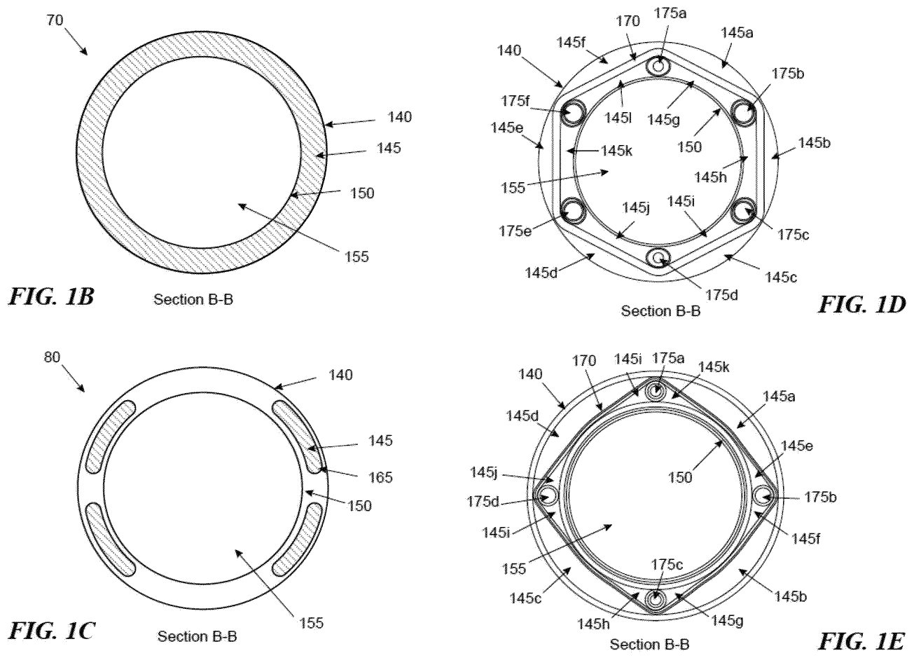

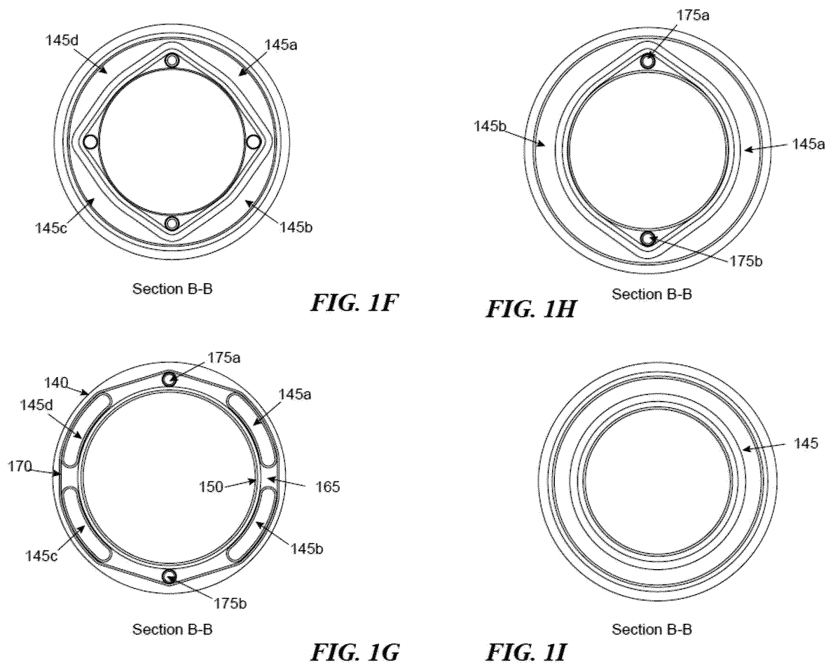

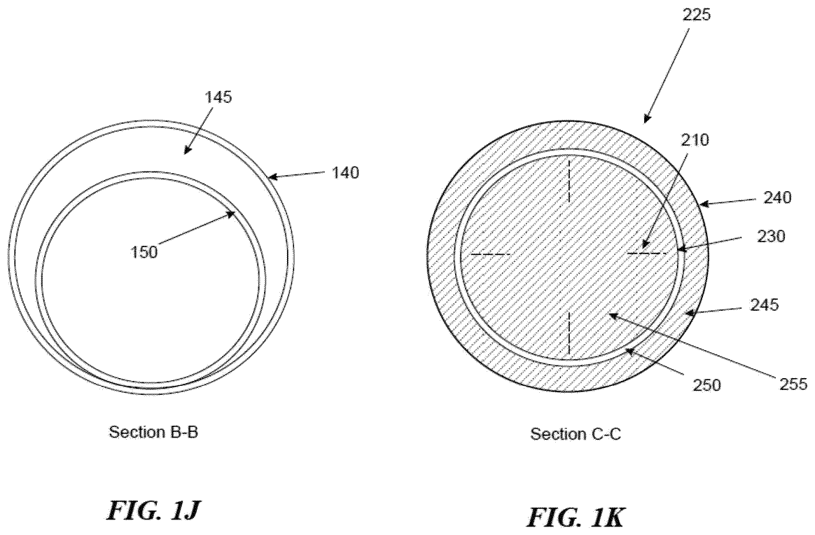

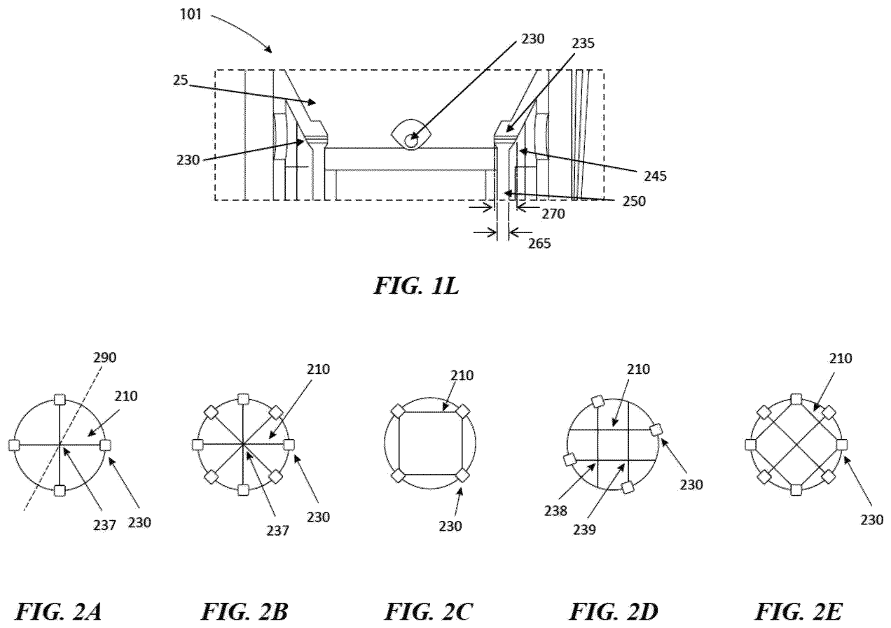

FIG. 1 illustrates a distal portion 10 of a thrombus removal system according to an embodiment of the present technology. FIG. 1A Section A-A illustrates an elevation sectional view of the distal portion. The example section A-A in FIG. 1A depicts a funnel 20 that is positioned at the distal end of the distal portion 10, the funnel adapted to engage with thrombus and/or a tissue (e.g., vessel) wall to aid in thrombus collection, fragmentation, and/or removal. The funnel can have a variety of shapes and constructions as would be understood by one of skill from the description herein. The example section A-A in FIG. 1A depicts a double walled thrombus removal device construction having an outer wall/tube 40 and an inner wall/tube 50. An aspiration lumen 55 is formed by the inner wall 50 and is centrally located. A generally annular volume forms at least one fluid lumen 45 between the outer wall 40 and the inner wall 50. The fluid lumen 45 is adapted for fluid communication with the fluid delivery mechanism. One or more apertures (e.g., nozzles, orifices, or ports) 30 are positioned in the thrombus removal system to be in fluid communication with the fluid lumen 45 and an irrigation manifold 25. In operation, the ports 30 are adapted to direct (e.g., pressurized) fluid toward thrombus that is engaged with the distal portion 10 of the thrombus removal system.

In various embodiments, the system can have an average flow velocity within the fluid lumen of up to 20 m/s to achieve consistent and successful aspiration of clots. In some embodiments, the fluid source itself can be delivered in a pulsed sequence or a preprogrammed sequence that includes some combination of pulsatile flow and constant flow to deliver fluid to the jets. In these embodiments, while the average pulsed fluid velocity may be up to 20 m/s, the peak fluid velocity in the lumen may be up to 30 m/s or more during the pulsing of the fluid source. In some embodiments, the jets or apertures have an aperture size ranging between 0.005″ to 0.020″ to avoid undesirable spraying of fluid. In some embodiments, the system can have a minimum vacuum or aspiration pressure of 15 inHg, to remove target clots after they have been macerated or broken up with the jets described above.

The thrombus removal system can be sized and configured to access and remove thrombi in various locations or vessels within a patient's body. It should be understood that while the dimensions of the system may vary depending on the target location, generally similar features and components described herein may be implemented in the thrombus removal system regardless of the application. For example, a thrombus removal system configured to remove pulmonary embolism (PE) from a patient may have an outer wall/tube with a size of approximately 11-13 Fr, or preferably 12 Fr, and an inner wall/tube with a size of 7-9 Fr, or preferably 8 Fr. A deep vein thrombosis (DVT) device, on the other hand, may have an outer wall/tube with a size of approximately 9-11 Fr, or preferably 10 Fr, and an inner wall/tube with a size of 6-9 Fr, or preferably 7.5 Fr. Applications are further provided for ischemic stroke and peripheral embolism applications.

Section B-B of FIG. 1B illustrates in plan view a portion of the thrombus removal system that is proximal to the funnel and irrigation manifold. Section B-B depicts an outer wall 140, an inner wall 150, an aspiration lumen 155 and a fluid lumen 145. In some embodiments, in cross-section the aspiration lumen 155 is generally circular and the fluid lumen 145 is generally annular in shape (e.g., cross-section 70). It will be appreciated that alternative constructions and/or arrangements of the inner wall 150 and the outer wall 140 produce variations in cross-sectional shape of the aspiration and fluid lumens 155 and 145. For example, the inner wall 150 can be shaped to form an aspiration lumen 155 that, in cross-section, is generally oval, circular, rectilinear, square, pentagonal, or hexagonal. The inner and outer walls 150 and 140 can be shaped and arranged to form a fluid lumen 145 that, in cross-section, is generally crescent-shaped, diamond shaped, or irregularly shaped. For example, referring to FIG. 1C Section B-B, the region between the inner wall 150 and the outer wall 140 can include one or more wall structures 165 that form respective fluid lumens 145 (e.g., as in cross-section 80). The wall structures 165 can be formed by lamination between the outer and inner walls 140 and 150, or by a multi-lumen extrusion that forms a plurality of the wall structures.

Section B-B of FIGS. 1D-1H illustrate additional examples of a portion of the thrombus removal system that is proximal to the funnel and irrigation manifold. Similar to the embodiments described above, the portion in these examples can include an outer wall 140, an inner wall 150, and an aspiration lumen 155. Additionally, the illustrated portion of the thrombus removal system can include a middle wall 170 disposed between the outer wall 140 and the inner wall 150. The middle wall 170 enables further segmentation of the annular space between the inner wall and outer wall into a plurality of distinct fluid lumens and/or auxiliary lumens. For example, referring to FIG. 1D, the middle wall can be generally hexagon shaped, and the annular space can include a plurality of fluid lumens 145a-141 and a plurality of auxiliary lumens 175a-175f. As shown in FIG. 1D, the fluid lumens can be formed by some combination of the outer wall 140 and the middle wall 170, or between the middle wall 170, the inner wall 150, and two of the auxiliary lumens. For example, fluid lumen 145a is formed in the space between outer wall 140 and middle wall 170. However, fluid lumen 145g is formed in the space between middle wall 170, inner wall 150, auxiliary lumen 175a, and auxiliary lumen 175b. Generally, the fluid lumens are configured to carry a flow of fluid such as saline from a saline source of the system to one or more ports/apertures/orifices of the system. The auxiliary lumens can be configured for a number of functions. In some embodiments, the auxiliary lumens can be coupled to the fluid/saline source and to the apertures to be used as additional fluid lumens. In other embodiments, the auxiliary lumens can be configured as steering ports and can include a guide wire or steering wire within the lumen for steering of the thrombus removal system. Additionally, in other embodiments, the auxiliary lumens can be configured to carry electrical, mechanical, or fluid connections to one or more sensors. For example, the system may include one or more electrical, optical, or fluid based sensors disposed along any length of the system. The sensors can be used during therapy to provide feedback for the system (e.g., sensors can be used to detect clogs to initiate a clog removal protocol, or to determine the proper therapy mode based on sensor feedback such as jet pulse sequences, aspiration sequences, and or proper functioning of the system, etc.). The auxiliary ports can therefore be used to connect to the sensors, e.g., by electrical connection, optical connection, mechanical/wire connection, and/or fluid connection. It is also contemplated that the fluid and auxiliary lumens can be configured to carry and deliver other fluids, such as thrombolytics or radio-opaque contrast injections to the target tissue site during treatment.

It should be understood that in some embodiments, all the fluid lumens are fluidly connected to all of the jets or apertures of the thrombus removal device. Therefore, when a flow of fluid is delivered from the fluid lumen(s) to the jets, all jets are activated with a jet of fluid at once. However, it should also be understood that in some embodiments, the fluid lumens are separate or distinct, and these distinct fluid lumens may be fluidly coupled to one or more jets but not to all jets of the device. In these embodiments, a subset of the jets can be controlled by delivering fluid only to the fluid lumens that are coupled to that subset of jets. This enables additional functionality in the device, in which specific jets can be activated in a user defined or predetermined order.

In various embodiments, the fluid pressure is generated at the pump (at the console or handle). The fluid is accelerated as it exits through the ports at the distal end and is directed to the target clot. In this way a wider variety of cost-effective components can be used to form the catheter while still maintaining a highly-effective device for clot removal. Additional details are provided below.

Section B-B of FIG. 1E illustrates another embodiment of the portion of the thrombus removal system that is proximal to the funnel and irrigation manifold. Similar to the embodiment of FIG. 1D, this embodiment also includes a middle wall 170. However, the middle wall in this example is generally square shaped, facilitating the formation of fluid lumens 145a-145k and auxiliary lumens 175a-175d. The example illustrated in section B-B of FIG. 1F is similar to that of the embodiment of FIG. 1E, however this embodiment includes only fluid lumens 145a-145d. The fluid lumens 145c-145k from the embodiment of FIG. 1E are not used as fluid lumens in this embodiment. They can be, for example, empty lumens, vacuum, filled with an insulative material, and/or filled with a radio-opaque material or any other material that may help visualize the thrombus removal system during therapy. The embodiment IF includes the same four auxiliary reports as illustrated and described in the embodiment of FIG. 1E.

Section B-B of FIG. 1G illustrates another example of a portion of the thrombus removal system that is proximal to the funnel and irrigation manifold. Similar to the embodiments described above, the illustrated portion of the thrombus removal system can include a middle wall 170 disposed between the outer wall 140 and the inner wall 150. However, this embodiment includes four distinct fluid lumens 145a-145d formed by wall structures 165. As with the embodiment of FIG. 1C, the wall structures 165 can be formed by lamination between the outer and inner walls 140 and 150, or by a multi-lumen extrusion that forms a plurality of the wall structures. As shown, this embodiment can include a pair of auxiliary lumens 175a and 175b, which can be used, for example, for steering or for sensor connections as described above.

Section B-B of FIG. 1H is another similar embodiment in which the middle wall and outer wall can be used to form fluid lumens 145a and 145b. Auxiliary lumens 175a and 175b can be formed in the space between the middle wall and the inner wall. It should be understood that the middle wall can contact the outer wall to create independent fluid lumens 145a and 145b. However, in other embodiments, it should be understood that the middle wall may not contact the outer wall, which would facilitate a single annular fluid lumen, such as is shown by fluid lumen 145 in Section B-B of FIG. 1I. In another embodiment, as shown in Section B-B of FIG. 1J, the inner wall 150 and the outer wall 140 may not be concentric, which facilitates formation of an annular space and/or fluid lumen 145 that is thicker or wider on one side of the device relative to the other side. As shown in FIG. 1J, a distance between the exemplary outer wall 140 and inner wall at the top (e.g., 12 o'clock) portion of the device is larger than a distance between the outer wall and inner wall at the bottom (e.g., 6 o'clock) portion of the device.

Section C-C of FIG. 1K illustrates in plan view a portion of the thrombus removal system comprising an irrigation manifold 225. Section C-C depicts an outer wall 240, an inner wall 250, a fluid lumen 245, an aspiration lumen 255, and ports 230 for directing respective fluid streams 210.

Detail View 101 of FIG. 1L illustrates a section view in elevation of a portion of the irrigation manifold 25 that includes a plurality of ports 230 that are formed within an inner wall 250. In some embodiments, a thickness of one or more walls of the thrombus removal system may be varied along its axial length and/or its circumference. As shown in Detail View 101, inner wall 250 has a first thickness 265 in a region 250 that is proximal to the irrigation manifold 25, and a second thickness 270 in a region 235 that includes the ports 230. In some embodiments, the second thickness 270 is greater than the first thickness 265. The first thickness 265 can correspond to a general wall thickness of the inner wall 50 and/or of the outer wall 40, which can be from about 0.10 mm to about 0.60 mm, or any value within the aforementioned range. The second thickness 270 can be from about 0.20 mm to about 0.70 mm, from about 0.70 mm to about 0.90 mm, or from about 0.90 mm to about 1.20 mm. The second thickness 270 can be any value within the aforementioned range. The dimension of the second thickness 270 can be selected to provide a fluid path through the ports 230 that produces a generally laminar flow for a fluid stream that is directed therethrough, when the fluid delivery mechanism supplies fluid via the fluid lumen 245 at a typical operating pressure. Such operating pressure can be from about 10 psi to about 60 psi, from about 60 psi to about 100 psi, or from about 100 psi to about 150 psi. The operating pressure of the fluid delivery mechanism can be any value within the aforementioned range of values. In some embodiments, the fluid delivery mechanism is operated in a high pressure mode, having a pressure from about 150 psi to about 250 psi, from about 250 psi to about 350 psi, from about 350 psi to about 425 psi, or from about 425 psi to about 500 psi, or up to 1,000 psi. The operating pressure of the fluid delivery mechanism in the high pressure mode can be any value within the aforementioned range of values.

The manifold is configured to increase a fluid pressure and/or flow rate of the fluid. When fluid is provided by the fluid delivery mechanism to the fluid lumen(s) at a first pressure and/or a first flow rate, the manifold is configured to increase the pressure of the fluid to a second pressure and/or is configured to increase the flow rate of the fluid to a second flow rate. The second pressure and/or second fluid rate can be higher than the first pressure and/or first flow rate. As a result, the manifold can be configured to increase the relatively low operating pressures and/or flow rates generated by the fluid delivery mechanism to the relatively high pressures and/or high flow rates generated by the ports/fluid streams.

In some embodiments, a profile (cross-sectional dimension) of a port 230 varies along its length (e.g., is non-cylindrical). A variation in the cross-sectional dimension of the port may alter and/or adjust a characteristic of fluid flow along the port 230. For example, a reduction in cross-sectional dimension may accelerate a flow of fluid through the port 230 (for a given volume of fluid). In some embodiments, a port 230 may be conical along its length (e.g., tapered), such that its smallest dimension is positioned at the distal end of the port 230, where distal is with respect to a direction of fluid flow.

In some embodiments, the port 230 is formed to direct the fluid flow along a selected path. FIGS. 2A-2E illustrate various embodiments of arrangements of ports 230 for directing respective fluid streams 210. In some embodiments, such as those shown in FIGS. 2A and 2B, at least two ports 230 are arranged to produce (e.g., respective) fluid streams 210 that intersect at an intersection region 237 of the thrombus removal system. An intersection region 237 can be a region of increased fluid momentum, turbulence, shear, and/or energy transfer, which multiply with respect to individual fluid streams that are not directed to combine at the intersection. The increased fluid momentum and/or energy transfer at an intersection may advantageously fragment thrombus more efficiently and/or quickly. As described above, the fluid streams can be configured to accelerate and cause cavitation and/or other effects to further add to breaking up of the target clot. In some embodiments, an intersection region can be formed from at least 2, at least 3, at least 4, at least 5, at least 6, at least 7, at least 8, at least 9, or at least 10 fluid streams 210. An intersection region can be generally near a central axis 290 of the thrombus removal system (e.g., 237), or away from the central axis (e.g., 238 and 239 in the embodiment of FIG. 2D). In some embodiments, at least two intersection regions (e.g., 238 and 239) are formed. In some embodiments, one or more ports 230 are arranged to direct a fluid stream 210 along an oblique angle with respect to the central axis of the thrombus removal system. An operating pressure of the fluid delivery mechanism may be selected to approach a minimum targeted fluid velocity for a fluid stream 210 that is delivered from a port 230. The targeted fluid velocity for a fluid stream 210 can be about 5 meters/second (m/s), about 8 m/s, about 10 m/s, about 12 m/s, or about 15 m/s. Additionally, the targeted fluid velocities in some embodiments can be in the range above 15 m/s to up to 150 m/s. At these higher velocities (e.g. above 15 m/s, or alternatively above 20 m/s), the fluid streams may be configured to generate cavitation in a target thrombus or tissue. It has been found that with fluid exiting from the ports to these flow rates a cavitation effect can be created in the focal area of the intersecting or colliding fluid streams, or additionally at a boundary of one or more of the fluid streams. While the exact specifications may change based on the catheter size, in general, at least one of the fluid streams should be accelerated to such a high velocity to create cavitation as described in detail below. The targeted fluid velocity for fluid stream 210 can be any value within the range of aforementioned values. In some embodiments, at least two ports 230 are adapted to deliver respective fluid streams at different fluid velocities (i.e. speed and direction), for a given pressure of the fluid delivery mechanism. In some embodiments, at least two ports 230 are adapted to deliver respective fluid streams at the substantially the same fluid velocities, for a given pressure of the fluid delivery mechanism. In some embodiments, one port is adapted to deliver fluid at high velocity and the respective one or more other ports is adapted to deliver fluid at relatively lower velocities. Advantageously, an increased cross-sectional area of the fluid lumen 145 reduces a required operating pressure of the fluid delivery mechanism to achieve a targeted fluid velocity of the fluid streams.

In some embodiments, the fluid streams are configured to create angular momentum that is imparted to a thrombus. In some examples, angular momentum is imparted on the thrombus by application of a) at least one fluid stream 210 that is directed at an oblique angle from a port 230, and/or b) at least two fluid streams 210 that have different fluid velocities. For example, fluid streams that cross near each other but do not necessarily intersect may create a “swirl” or rotational energy on the clot material. Advantageously, angular momentum produced in a thrombus may impart a (e.g., centrifugal) force that assists in fragmentation and removal of the thrombus. Rotating of the clot may enhance delivery of the clot material to the jets. By example, with a large, amorphous clot the soft material may be easily aspirated or broken up by the fluid streams whereas tough fibrin may be positioned away from the fluid streams. Rotating or swirling of the clot moves the material around so the harder clot material is presented to the jets. The swirling may also further break up the clot as it is banged inside the funnel.

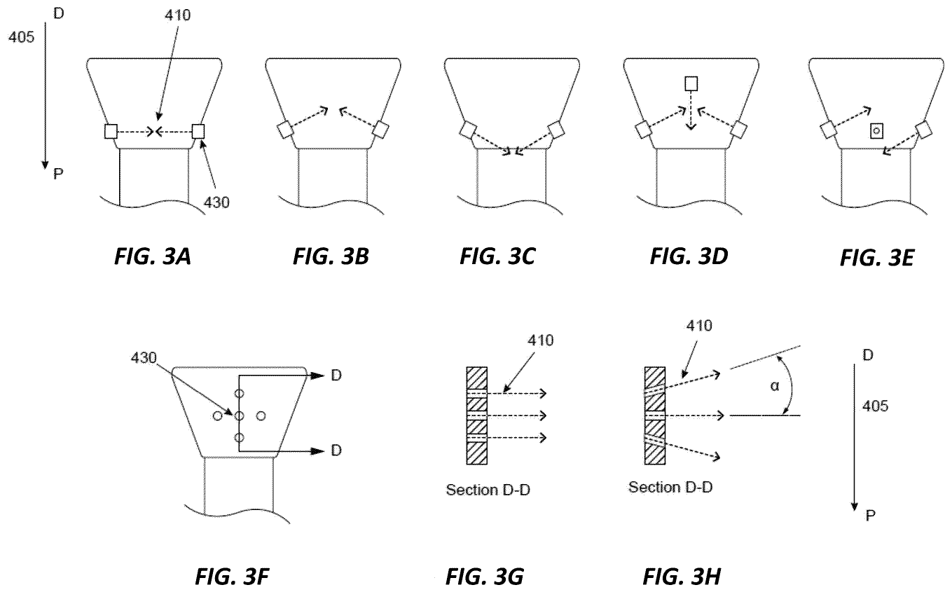

FIGS. 3A-3H depict various configurations of fluid streams 410 that are directed from respective ports 430. A fluid stream 410 can be directed along a path that is substantially orthogonal, proximal, and/or distal to the flow axis 405 (which is like to flow axis 305). In some embodiments, at least two fluid streams are directed in different directions with respect to the flow axis 405. In some embodiments, at least two fluid streams are directed in a same direction (e.g., proximally) with respect to the flow axis 405. In some embodiments, at least a first fluid stream is directed orthogonally, at least a second fluid stream is directed proximally, and at least a third fluid stream is directed distally with respect to the flow axis 405. An angle α may characterize an angle that a fluid stream 410 is directed with respect to an axis that is orthogonal to the flow axis 405 (e.g., as shown in section D-D of FIGS. 3G and 3H). An intersection region of fluid streams can be within an interior portion of the thrombus removal system, and/or exterior (e.g., distal) to the thrombus removal system. In some embodiments, a fluid stream that is directed by a port 430 in a nominal direction (e.g., distally) is deflected along an altered path (e.g., proximally) by (e.g., suction) pressure generated by the aspiration mechanism during operation.

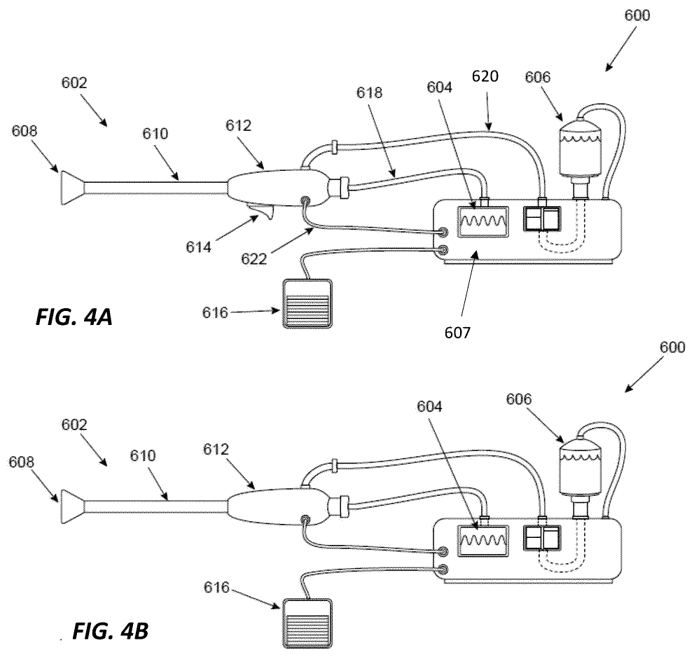

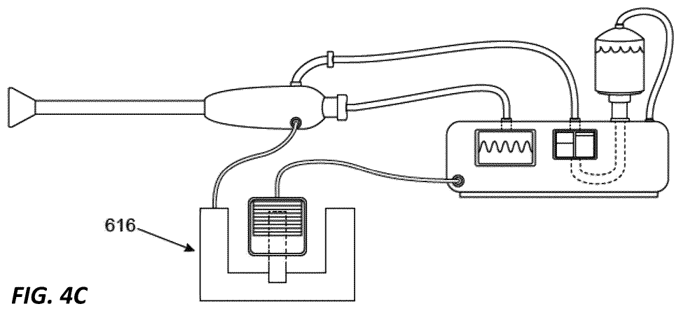

FIGS. 4A-4C illustrate various configurations of a thrombus removal system 600, including a thrombus removal device, 602, a vacuum source and cannister 604, and a fluid source 606. In some embodiments, the vacuum source and cannister and the fluid source are housed in a console unit that is detachably connected to the thrombus removal device. A fluid pump can be housed in the console, or alternatively, in the handle of the device. The console can include one or more CPUs, electronic controllers, or microcontrollers configured to control all functions of the system. The thrombus removal device 602 can include a funnel 608, a flexible shaft 610, a handle 612, and one or more controls 614 and 616. For example, in the embodiment shown in FIG. 4A, the device can include a finger switch or trigger 614 and a foot pedal or switch 616. These can be used to control aspiration and irrigation, respectively. Alternatively, as shown in the embodiment of FIG. 4B, the device can include only a foot switch 614, which can be used to control both functions, or in FIG. 4C, the device can include only an overpedal 616, also used to control both functions. It is also contemplated that an embodiment could include only a finger switch to control both aspiration and irrigation functions. As shown in FIG. 4A, the vacuum source can be coupled to the aspiration lumen of the device with a vacuum line 618. Any clots or other debris removed from a patient during therapy can be stored in the vacuum cannister 604. Similarly, the fluid source (e.g., a saline bag) can be coupled to the fluid lumens of the device with a fluid line 620.

Still referring to FIG. 4A, electronics line 622 can couple any electronics/sensors, etc. from the device to the console/controllers of the system. The system console including the CPUs/electronic controllers can be configured to monitor fluid and pressure levels and adjust them automatically or in real-time as needed. In some embodiments, the CPUs/electronic controllers are configured to control the vacuum and irrigation as well as electromechanically stop and start both systems in response to sensor data, such as pressure data, flow data, etc.

As is described above, aspiration occurs down the central lumen of the device and is provided by a vacuum pump in the console. The vacuum pump can include a container that collects any thrombus or debris removed from the patient.

### PV Loop Measurements

Systems and methods are also provided herein for performing right ventricular pressure-volume (PV) analysis before, during, and/or after thrombectomy procedures to characterize ventricular systolic and diastolic properties independent of loading conditions and assess procedure completion. The systems and methods herein can use a correlation of pulmonary artery (PA) vascular function and right ventricular (RV) function with this PV loop analysis to inform a physician of treatment progress and/or treatment completion.

Thrombectomy systems provided herein can include the system components described above, including a thrombectomy catheter that may include a flexible shaft, a distal expandable funnel, an aspiration lumen coupled to an aspiration source, and optionally two or more fluid apertures for producing jets or fluid streams at or within the distal expandable funnel. The system can further include a delivery system configured to delivery and position the thrombectomy catheter at a target location, such as within the pulmonary artery in proximity to one or more pulmonary embolisms or clots. The delivery system can include a guidewire, an introducer catheter or sheath and a dilator. In some aspects, the introducer catheter can include one or more sensors such as pressure sensors configured to measure parameters of the patient (e.g., pulmonary artery pressure).

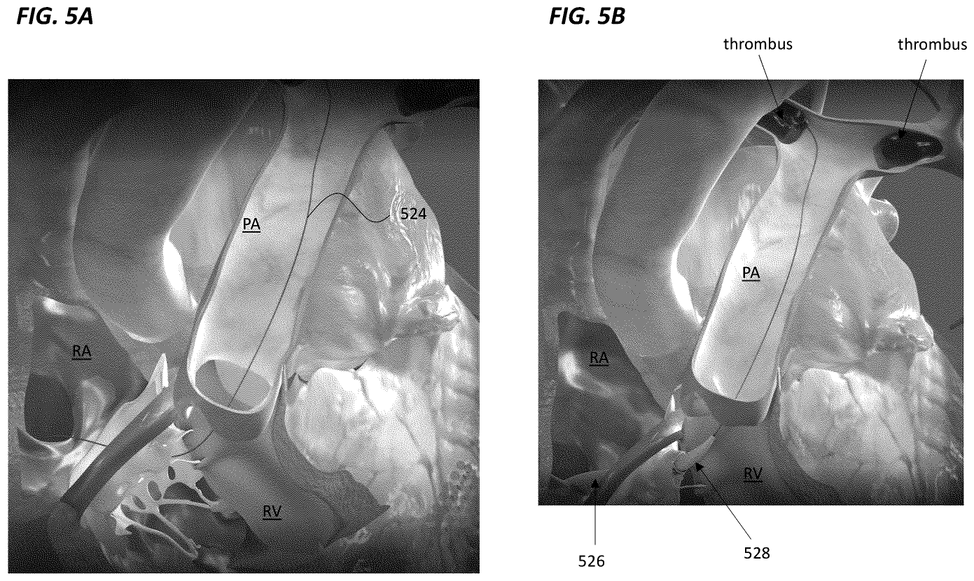

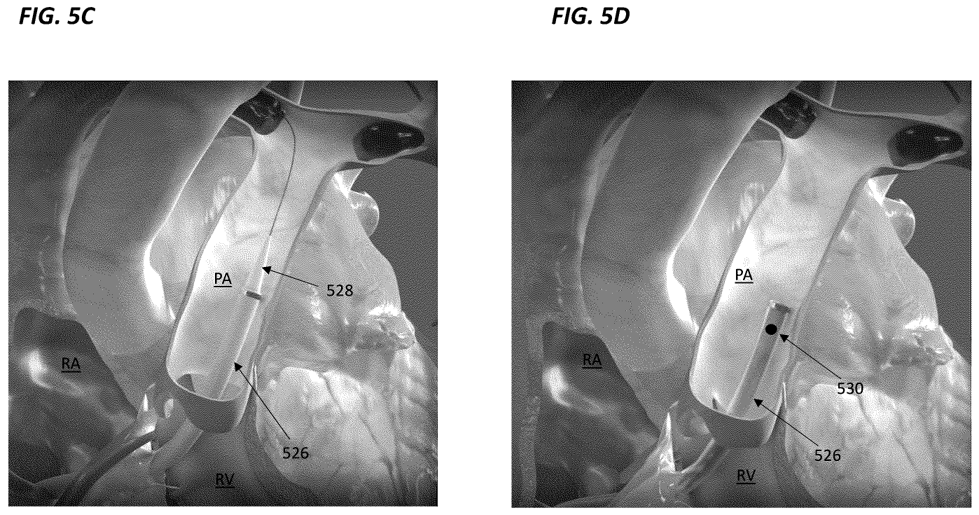

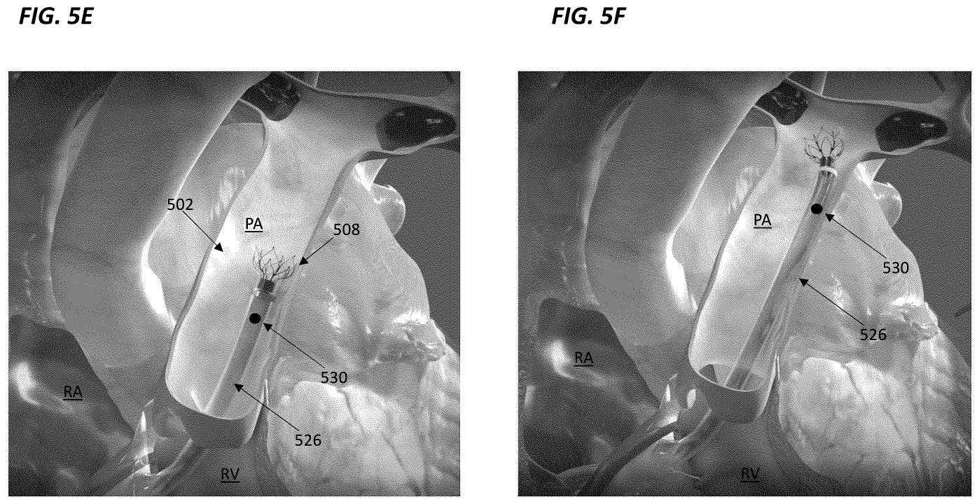

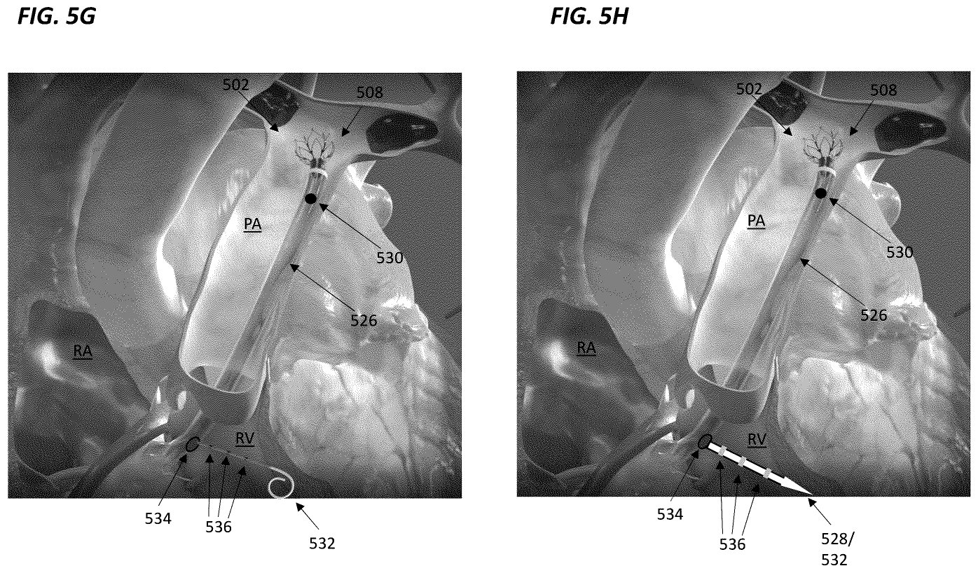

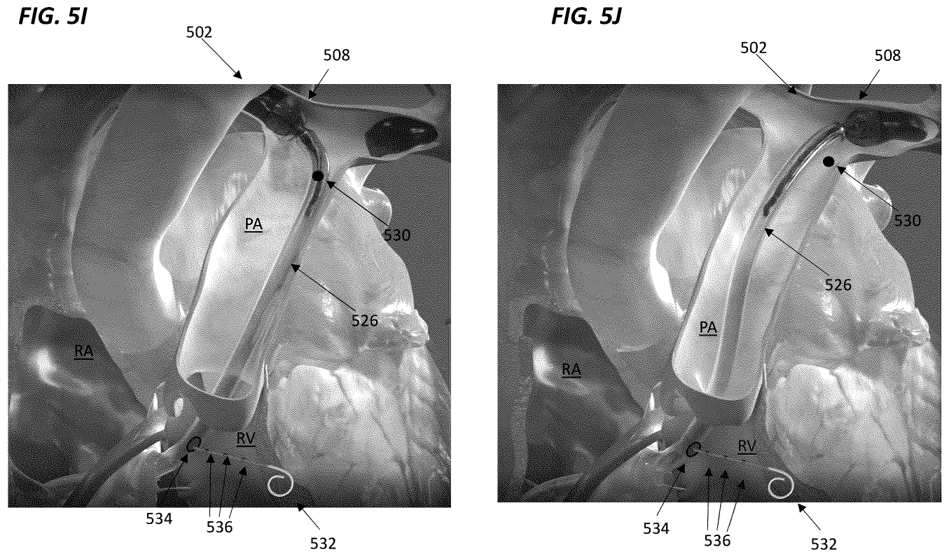

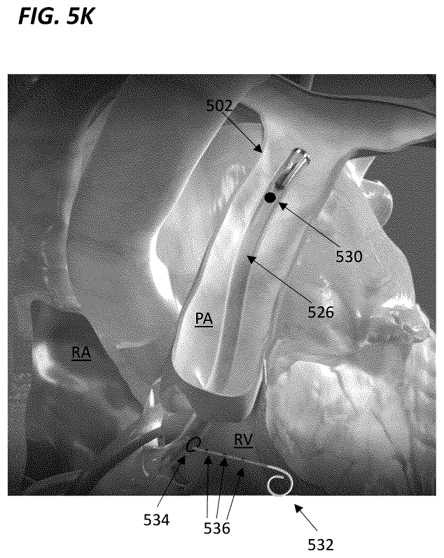

FIGS. 5A-5K illustrate a thrombectomy method that can include performing PV loop analysis. The PV loop analysis can be used to determine treatment progress and/or completion.

In FIG. 5A, a guidewire 524 can be advanced into the right atrium (RA), through the right ventricle (RV), and into the pulmonary artery (PA). Typically a femoral approach is used, however other approaches such as the jugular approach can also be considered. In FIG. 5B, at least one thrombus is shown in the PA, and an introducer catheter/sheath 526 and dilator 528 are advanced over the guidewire. Here, the dilator is shown extending into the RV.

FIG. 5C shows the introducer sheath 526 and dilator 528 advanced into the PA, proximal to the targeted thrombi. In FIG. 5D, the dilator can be retracted proximally into the sheath and optionally out of the patient, leaving only the introducer sheath in the PA. In FIG. 5D, it is also shown that the introducer sheath 526 can include a pressure sensor 530. The pressure sensor can comprise, for example, a fiber optic pressure sensor. In some embodiments, the pressure sensor is integrated into the sheath to measure a blood pressure parameter in the patient. When the introducer sheath is positioned in the PA, the pressure sensor can be configured to measure pulmonary artery pressure. Any pressure sensor known in the art can be integrated into the introducer sheath, either externally or internally to the introducer sheath.

In FIG. 5F, a thrombectomy catheter 502, such as any of the thrombectomy catheters or devices described herein, can be inserted into the introducer catheter 526 and advanced into the pulmonary artery. When the thrombectomy catheter 502 is advanced distally beyond the distal end of the introducer sheath 526, an expandable funnel 508 of the thrombectomy catheter can assume an expanded configuration as shown. In FIG. 5F, the introducer sheath and thrombectomy catheter can be further advanced and positioned adjacent to the one or more thrombi. Proper positioning can be confirmed with real-time imaging and/or contrast injections into the vasculature (e.g., by injecting contrast through the introducer sheath into the pulmonary artery in the directions of the target thrombi. The pressure sensor 530 of the introducer sheath can continuously or periodically measure pressure in the pulmonary artery when the thrombectomy catheter is in position. In some embodiments, a user or physician of the system can provide an input to the system (e.g., press a button) to take an on-demand pressure sensor measurement.

Referring to FIGS. 5G and 5H, in some embodiments a pressure-volume conductance catheter (P-V catheter) 532 can be advanced out of a port 534 in the introducer sheath 526 into the RV. A PV catheter can be configured to pass a high-frequency low-amplitude current through two or more sets of electrodes 536 along a length of the RV and simultaneously measure electrical potentials that are proportional to ventricular volume. With calibration, these signals can be converted to instantaneous RV blood volume measurements. The P-V catheter can further include a pressure transducer (not shown) allowing real time pressure-volume loop generation.

In the example of FIG. 5G, the P-V catheter 532 can comprise a catheter that is separate from the thrombectomy catheter 502. In one example, the P-V catheter 532 can be advanced within the introducer sheath 526 to port 534, where the catheter can exit the sheath into the RV when the port 534 is positioned in or near the RV. In some examples, the introducer sheath 526 can include more than one lumen, for example, a first lumen for the dilator and thrombectomy catheter and a second lumen for the P-V catheter. The first lumen can terminate at the distal end of the sheath, and the second lumen can terminate at the port 534. In another embodiment, the sheath can include only a single lumen and both the thrombectomy catheter and the P-V catheter can be advanced side-by-side to the appropriate position. In another example, the P-V catheter can be built-in or integrated into the introducer sheath. In yet another example, the P-V catheter can be placed via a separate vascular approach (e.g., via a jugular approach when the introducer sheath uses a femoral approach).

In the embodiment of FIG. 5H, instead of using a separate P-V catheter, the dilator 528 can be retrofit or modified to perform the function of a P-V catheter 532. For example, the dilator can include electrodes 536 configured to pass a high-frequency low-amplitude current along a length of the RV and simultaneously measure electrical potentials that are proportional to ventricular volume. These signals can be converted to instantaneous RV blood volume measurements. The dilator 528 can further include a pressure transducer allowing real time pressure-volume loop generation. In this example, the dilator can still perform the introducing function described above in FIGS. 5B-5C, and then the dilator can be retracted into the introducer sheath 526 and advanced out of port 534 in the RV.

In the embodiments of FIGS. 5G-5H, the pressure sensor 530 can measure PA pressure, and the P-V catheter can measure conductance/admittance in the RV and also measure pressure in the RV. As described above, the P-V catheter can further perform a pressure-volume loop with the measured data. Before attempting to remove the clot(s) with the thrombectomy catheter, the pressure, conductance, and/or admittance measurements can provide a baseline characterization of vascular function and RV function prior to a thrombectomy procedure.

In FIG. 5I, the thrombectomy catheter 502 can be advanced towards a clot. In some examples, aspiration can be activated on the thrombectomy catheter to engage with the clot in the funnel. Optionally, the thrombectomy catheter can administer jets or fluid streams into the clot to help break up and remove the clot via the catheter aspiration. The pressure sensor 530 and P-V catheter 532 can continuously or periodically obtain pressure, conductance, and/or admittance measurements during the thrombectomy procedure, including prior to engagement with the clot, during engagement with the clot, and removal of the clot. In some examples, as described above, the P-V catheter measurements can be used to perform a pressure-volume loop analysis.

FIG. 5J shows continued clot removal, this time with the thrombectomy catheter 502 moved to a new clot location and engaged with the clot. As described above, the pressure sensor 530 and P-V catheter 532 can continuously or periodically obtain pressure, conductance, and/or admittance measurements during the thrombectomy procedure, including prior to engagement with the clot, during engagement with the clot, and removal of the clot. In some examples, as described above, the P-V catheter measurements can be used to perform a pressure-volume loop analysis.

FIG. 5K shows the introducer sheath still inserted into the PA after removal of the two target clots from FIGS. 51 and 5J. In this example, the thrombectomy catheter 502 can be seen retracted into the sheath but still positioned near the distal end of the sheath. After the clots have been removed, the pressure sensor 530 and P-V catheter 532 can continuously or periodically obtain pressure, conductance, and/or admittance measurements during the thrombectomy procedure, including prior to engagement with the clot, during engagement with the clot, and removal of the clot. In some examples, as described above, the P-V catheter measurements can be used to perform a pressure-volume loop analysis.

The thrombectomy system described herein and above in FIGS. 5A-5K can employ algorithms and software to determine a correlation between sensed parameters such as PA pressure, measurements in the RV including conductance, admittance, RV pressure, and/or pressure-volume loop analysis, and treatment progress or completion. For example, the correlation can use the measurements from the system to determine or quantify how the treatment is progressing. In some examples, the correlation can be between vascular function and RV function. In some examples, the treatment progress/completion is algorithmically determined (e.g., a change in the PA pressure, change in RV function, change in conductance/admittance, and/or the pressure-volume loop analysis exceeds a pre-determined threshold) and the user or physician can be notified or alerted of a treatment progression state or system state (e.g., with an alert, light, indicator, or icon on a display, or with a sound from the system console). For example, a change in one or more of the measured parameters that exceeds a first threshold may indicate that a first (of one or more) clots has been removed. Alternatively, a change in one or more of the measured parameters that exceeds a second threshold may indicate treatment completion and return to normal or acceptable vascular and/or RV function.

Alternatively, the thrombectomy system can employ machine learning and/or artificial intelligence (AI) to determine or indicate treatment progress or completion based on the PA and/or RV measurements, including PA pressure, RV conductance, RV admittance, and/or the pressure-volume loop analysis. The PA and/or RV measurements can be input into a trained machine learning model, and the model can output a label or determination as to the progress or completion of the thrombectomy procedure. For example, the machine learning model may use the correlation between vascular function and RV function to determine treatment progress or completion. The output can comprise, for example, a descriptor of treatment progress (e.g., untreated, partially treated, treatment completion, etc.), or a descriptor of clot status (e.g., clot removed, clot(s) remaining, clot(s) cleared, etc.). The output can also comprise an indication of treatment progress (e.g., 10% treated, 50% treated, 75% treated, 100% treated, etc.).

The machine learning model(s) can be trained by tagging treatment progress/completion state while onboard data and/or the PA and RV measurements are obtained during a procedure. For example, a user can tag a treatment state of thrombus removed, thrombus partially removed, or thrombus not removed during a procedure, and the machine learning model can be trained to correlate onboard data such as aspiration pressure curves or measured data such as PA pressure, RV conductance, RV admittance, and/or pressure-volume loop analysis with a given treatment progress state (e.g., note treated, partially treated, treatment complete, etc.). The trained model can then be used as described above to determine treatment progress or completion during a procedure.

The thrombectomy systems described herein can further use a trained machine learning model or AI can be used to determine or characterize system state during a thrombectomy procedure, including 1) clear (funnel not engaged with clot), 2) partially engaged (funnel is partially but not fully engaged with clot), and 3) fully engaged/clogged. The trained machine learning model can use measured parameters and/or onboard data (e.g., aspiration pressure curves) to determine system state in concert with labeled training data.

In some implementations, the machine learning model(s) can be trained by tagging a system state while the onboard data and/or PA and RV measurements are obtained during a procedure. For example, a user can tag a system state of clear, partially engaged, or engaged/clogged during a procedure, and the machine learning model can be trained to correlate onboard data such as aspiration pressure curves or measured data such as PA pressure, RV conductance, RV admittance, and/or pressure-volume loop analysis with a given system state. The trained model can then be used as described above to determine system state during a procedure.

An example of an Al model may include a convolutional neural network relating to a U-net. A U-net may be a type of convolutional neural network used for data processing, according to any method described herein. A thrombectomy system may have a computer-based system operating the AI model, according to any method described herein. The AI model may process one or more data inputs into a first layer of the convolutional neural network (e.g., the U-net). The data may be processed through a series of layers. The processing layers of the AI model may be considered in one or more phases or paths of the data processing.

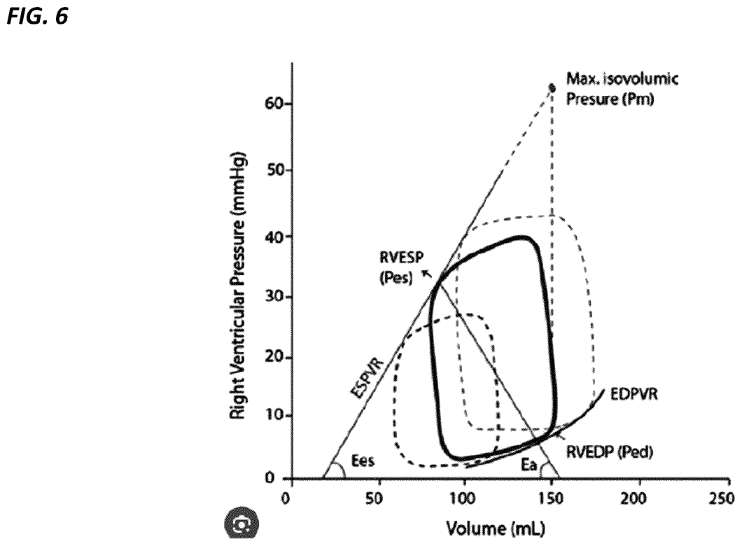

FIG. 6 is an example of a pressure-volume loop analysis that shows a correlation between right ventricular pressure and volume. The pressure-volume loop analysis can be generated by real-time measurement of pressure and volume within the right ventricle. Several physiologically relevant hemodynamic parameters such as stroke volume, cardiac output, ejection fraction, myocardial contractility, etc. can be determined from these loops.

### CONCLUSION

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

What is claimed is:

1. A method, comprising:
advancing a thrombectomy system into a pulmonary artery of a subject;
periodically or continuously measuring a pulmonary artery pressure with the thrombectomy system;
advancing a P-V catheter into a right ventricle of the subject;
periodically or continuously measuring right ventricular pressure, right ventricular conductance, and/or a right ventricular admittance with the P-V catheter;
initiating a thrombectomy procedure with the thrombectomy system; and
determining a treatment progress or treatment completion state based on a correlation between the measured pulmonary artery pressure and/or the right ventricular pressure, right ventricular conductance, and/or a right ventricular admittance.

2. The method of claim 1, further comprising providing an output related to the treatment progress or treatment completion state to a user of the thrombectomy system.

3. The method of claim 1, further comprising performing a pressure-volume loop analysis with the right ventricular pressure, right ventricular conductance, and/or a right ventricular admittance.

4. The method of claim 1, wherein the correlation is based on a relationship between vascular function and right ventricular function.

5. The method of claim 1, further comprising determining the treatment progress or treatment completion state algorithmically based on a change in a measured parameter exceeding a predetermined threshold.

6. The method of claim 1, further comprising determining the treatment progress or treatment completion state with a machine learning model.

7. The method of claim 6, wherein the machine learning model is trained by tagging the treatment progress or treatment completion state with one or more training data sets.

8. The method of claim 2, wherein the output comprises a label of treatment progress.

9. The method of claim 8, wherein the label is selected from the group consisting of not complete, partially complete, and treatment complete.

10. The method of claim 8, wherein the label is an indicator of treatment progress.

11. The method of claim 10, wherein the label is a percentage of treatment completion.

12. A method, comprising:
advancing a thrombectomy system into a pulmonary artery of a subject;
periodically or continuously measuring a pulmonary artery pressure with the thrombectomy system;
advancing a P-V catheter into a right ventricle of the subject;
periodically or continuously measuring right ventricular pressure, right ventricular conductance, and/or a right ventricular admittance with the P-V catheter;
initiating a thrombectomy procedure with the thrombectomy system;
evaluating onboard catheter data; and
determining a system state based on a correlation between the onboard data and/or measured pulmonary artery pressure and/or the right ventricular pressure, right ventricular conductance, and/or a right ventricular admittance.

13. The method of claim 12, further comprising providing an output of the system state to a user of the thrombectomy system.

14. The method of claim 12, further comprising performing a pressure-volume loop analysis with the right ventricular pressure, right ventricular conductance, and/or a right ventricular admittance.

15. The method of claim 12, wherein the correlation is based on a relationship between vascular function and right ventricular function.

16. The method of claim 12, further comprising determining the system state algorithmically based on a change in onboard catheter data exceeding a predetermined threshold.

17. The method of claim 12, further comprising determining the system state with a machine learning model.

18. The method of claim 17, wherein the machine learning model is trained by tagging the system state with one or more training data sets.

19. The method of claim 13, wherein the system state comprises a label that describes clot engagement.

20. The method of claim 19, wherein the label is selected from the group consisting of clear, partially engaged, and engaged.

21. A thrombectomy system, comprising:
an introducer sheath;
a thrombectomy device adapted to be inserted into the introducer sheath to place the thrombectomy device within a pulmonary artery of a subject, the thrombectomy device including an aspiration lumen coupled to an aspiration source;
a pressure sensor disposed on the introducer sheath and/or the thrombectomy device and configured to measure a pulmonary artery pressure of the subject;
a P-V catheter adapted to be inserted into a right ventricle of the subject, the P-V catheter being configured to measure a right ventricular pressure, a right ventricular conductance, and/or a right ventricular admittance;
one or more processors; and
memory coupled to the one or more processors, the memory configured to store computer-program instructions, that, when executed by the one or more processors, implement a computer-implemented method, the computer-implemented method comprising:
determining a treatment progress or treatment completion state based on a correlation between the measured pulmonary artery pressure and/or the right ventricular pressure, right ventricular conductance, and/or a right ventricular admittance.
